(19) United States
(12) Patent Application Publication
(10) Pub. No.: US 2019/0088362 A1
(43) Pub. Date: Mar. 21, 2019

(54) METHOD, SYSTEM, AND NON-TRANSITORY COMPUTER-READABLE RECORDING MEDIUM FOR PROVIDING MEDICAL SERVICE

(71) Applicant: Hyung Tay RHO, Seoul (KR)

(72) Inventor: Hyung Tay RHO, Seoul (KR)

(21) Appl. No.: 16/163,618

(22) Filed: Oct. 18, 2018

Related U.S. Application Data
(63) Continuation of application No. PCT/KR2017/004120, filed on Apr. 17, 2017.

(30) Foreign Application Priority Data
Apr. 18, 2016 (KR) 10-2016-0047180

(51) Int. Cl. G16H 40/20; G16H 80/00

(52) U.S. Cl. CPC G16H 40/20; G16H 80/00

(57) ABSTRACT

The present invention relates to a method, system, and non-transitory computer-readable recording medium for providing a medical service. According to one aspect of the invention, there is provided a method for providing a medical service, comprising the steps of: determining an in-hospital status and a necessary medical service of a patient visiting a hospital, with reference to information on a location of the patient and information on a medical appointment or an electronic medical record (EMR) of the patient; and dynamically matching the patient with at least one medical attendant or assistant medical attendant belonging to the hospital, with reference to information on at least one of the in-hospital status of the patient, the necessary medical service of the patient, an in-hospital status of the medical attendant or assistant medical attendant, and a providable medical service of the medical attendant or assistant medical attendant.

### CROSS-REFERENCE TO RELATED APPLICATION(S)

This application is a continuation application of Patent Cooperation Treaty (PCT) International Application Serial No. PCT/KR2017/004120, filed on Apr. 17, 2017, which claims priority to Korean Patent Application Serial No. 10-2016-0047180, filed on Apr. 18, 2016. The entire contents of PCT International Application Serial No. PCT/KR2017/004120 and Korean Patent Application Serial No. 10-2016-0047180 are hereby incorporated by reference.

### FIELD OF THE INVENTION

The present invention relates to a method, system, and non-transitory computer-readable recording medium for providing a medical service.

### BACKGROUND

Conventionally, medical resources of a hospital or a clinic have been managed commonly on the basis of intentional manipulation according to subjective judgment of a medical attendant or an assistant medical attendant, or on the basis of an active request of a patient. For example, a medical attendant or a bed can be allocated to a patient visiting a hospital only after the patient has completed a reception procedure, and treatment of the patient can proceed only after the medical attendant personally reads information on a medical appointment or an electronic medical record of the patient and recognizes the contents thereof.

However, according to the above conventional method, there is a limitation that medical attendants or assistant medical attendants cannot be properly and efficiently allocated in a small clinic with deficient medical attendants, or in a large hospital visited by a large number of patients, and there is also a problem that patients may be dissatisfied with lengthened waiting times.

In order to solve the above problems, a technique has been introduced in which, when a patient completes a reception procedure or the like through a counter or kiosk, notification information thereon can be automatically transmitted to a medical attendant. However, even with the above technique, there is a limitation in increasing the efficiency of allocating medical attendants, because a positive action or request of the patient and judgment or instruction of the medical attendant should be still involved in order for medical attendants or assistant medical attendants to substantially progress tasks for treatment of the patient.

In this regard, the present inventor(s) suggest a technique that may efficiently match a patient with a medical attendant using information on a status and a necessary medical service of the patient and information on a status and a providable medical service of the medical attendant.

### SUMMARY OF THE INVENTION

One object of the present invention is to solve all the above-described problems in the prior art.

Another object of the invention is to efficiently match a patient with a medical attendant using information on a status and a necessary medical service of the patient and information on a status and a providable medical service of the medical attendant, by determining an in-hospital status and a necessary medical service of a patient visiting a hospital, with reference to information on a location of the patient and information on a medical appointment or an electronic medical record (EMR) of the patient; and dynamically matching the patient with at least one medical attendant or assistant medical attendant belonging to the hospital, with reference to information on at least one of the in-hospital status of the patient, the necessary medical service of the patient, an in-hospital status of the medical attendant or assistant medical attendant, and a providable medical service of the medical attendant or assistant medical attendant.

The representative configurations of the invention to achieve the above objects are described below.

According to one aspect of the invention, there is provided a method for providing a medical service, comprising the steps of: determining an in-hospital status and a necessary medical service of a patient visiting a hospital, with reference to information on a location of the patient and information on a medical appointment or an electronic medical record (EMR) of the patient; and dynamically matching the patient with at least one medical attendant or assistant medical attendant belonging to the hospital, with reference to information on at least one of the in-hospital status of the patient, the necessary medical service of the patient, an in-hospital status of the medical attendant or assistant medical attendant, and a providable medical service of the medical attendant or assistant medical attendant.

According to another aspect of the invention, there is provided a system for providing a medical service, comprising: a patient monitoring unit configured to determine an in-hospital status and a necessary medical service of a patient visiting a hospital, with reference to information on a location of the patient and information on a medical appointment or an electronic medical record (EMR) of the patient; and a matching management unit configured to dynamically match the patient with at least one medical attendant or assistant medical attendant belonging to the hospital, with reference to information on at least one of the in-hospital status of the patient, the necessary medical service of the patient, an in-hospital status of the medical attendant or assistant medical attendant, and a providable medical service of the medical attendant or assistant medical attendant.

In addition, there are further provided other methods and systems to implement the invention, as well as non-transitory computer-readable recording media having stored thereon computer programs for executing the methods.

According to the invention, it is possible to efficiently match a patient with a medical attendant using information on an in-hospital status and a necessary medical service of the patient, which is determined on the basis of a location and the like of the patient, and information on an in-hospital status and a providable medical service of the medical attendant, which is determined on the basis of a location and the like of the medical attendant, thereby drastically reducing treatment waiting times of patients visiting a hospital, and increasing satisfaction and confidence of the patients in medical services.

According to the invention, it is possible to drastically reduce the time or effort (e.g., emotional labor) that medical attendants or assistant medical attendants should take to address dissatisfaction of patients due to waiting times.

According to the invention, it is possible to match a patient with a medical attendant in a direction suitable for situations of both the patient and the medical attendant, without needing the medical attendant to take additional time and effort to recognize the situation of the patient or other medical attendant, thereby increasing the work efficiency of medical attendants providing medical services.

According to the invention, it is possible to automatically recognize a treatment pattern of a medical attendant using a deep learning-based artificial intelligence algorithm or the like, so that a treatment schedule may be established as efficiently as possible within the capacity of the medical attendant, thereby improving sales of a medical institution and enhancing concentration and quality of treatment provided by doctors.

According to the invention, it is possible to predict a change in sales of a medical institution from a treatment schedule specified on the basis of a state of matching between a patient and a medical attendant, and to provide information on the prediction to management staff of the medical institution, thereby substantially assisting management of the medical institution.

According to the invention, it is possible to directly match patients and medical attendants to lower the dependence on management personnel required for establishing treatment schedules or managing working status of medical attendants, and to prevent the work efficiency of a medical institution from being greatly influenced by the skill level of the management personnel, thereby saving expenses due to the reduced management personnel and allowing the medical institution to be operated sustainably and efficiently.

According to the invention, it is possible to provide information on a treatment pattern of a medical attendant or other medical attendant for each subdivided treatment item, information on deficient treatment items of the medical attendant as compared to other medical attendants, information on new medical knowledge published at an academic conference or seminar, and the like, thereby enhancing concentration and competitiveness of treatment provided by the medical attendants.

### BRIEF DESCRIPTION OF THE DRAWINGS

FIG. 1 schematically shows the configuration of an entire system for providing a medical service according to one embodiment of the invention.

FIG. 2 specifically shows the internal configuration of a main system according to one embodiment of the invention.

FIG. 3A illustratively shows information provided to a device of a medical attendant or assistant medical attendant according to one embodiment of the invention.

FIG. 3B illustratively shows information provided to a device of a medical attendant or assistant medical attendant according to one embodiment of the invention.

FIG. 3C illustratively shows information provided to a device of a medical attendant or assistant medical attendant according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the locations or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the present invention will be described in detail with reference to the accompanying drawings to enable those skilled in the art to easily implement the invention.

Configuration of the Entire System

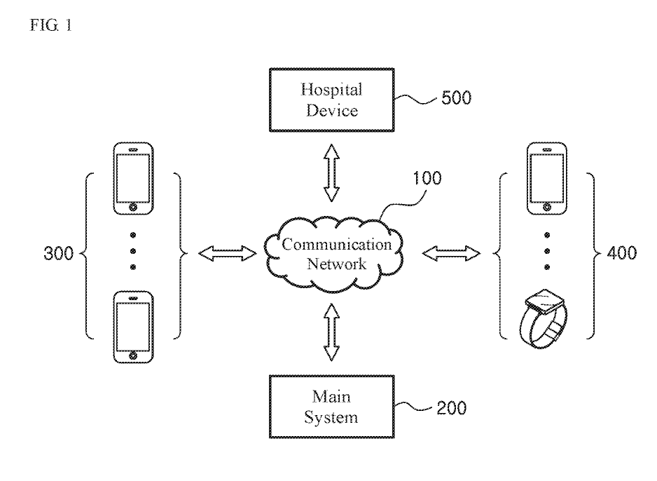

FIG. 1 schematically shows the configuration of an entire system for providing a medical service according to one embodiment of the invention.

As shown in FIG. 1, the entire system according to one embodiment of the invention may comprise a communication network 100, a main system 200, a patient device 300, a medical attendant device 400, and a hospital device (or hospital server) 500.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may be the Internet or the World Wide Web (WWW). However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, Long Term Evolution (LTE) communication, Bluetooth communication (more specifically, Bluetooth Low Energy (BLE) communication), infrared communication, and ultrasonic communication.

Next, according to one embodiment of the invention, the main system 200 may interwork with the patient device 300, the medical attendant device 400, or the hospital device (or hospital server) 500 on the communication network 100, and may function to efficiently match a patient with a medical attendant using information on a location and a necessary medical service of the patient and information on a location and a providable medical service of the medical attendant, by determining an in-hospital status and a necessary medical service of a patient visiting a hospital, with reference to information on a location of the patient and information on a medical appointment or an electronic medical record (EMR) of the patient; and dynamically matching the patient with at least one medical attendant or assistant medical attendant belonging to the hospital, with reference to information on at least one of the in-hospital status of the patient, the necessary medical service of the patient, an in-hospital status of the medical attendant or assistant medical attendant, and a providable medical service of the medical attendant or assistant medical attendant.

The configuration and function of the main system 200 according to the invention will be discussed in more detail below.

Next, according to one embodiment of the invention, the patient device 300 is digital equipment that may function to connect to and then communicate with the main system 200 or the hospital device (or hospital server) 500, and any type of digital equipment having a memory means and a microprocessor for computing capabilities, such as a smart phone and a tablet PC, may be adopted as the patient device 300 according to the invention.

Specifically, according to one embodiment of the invention, the patient device 300 may include a communication module (not shown) for wireless communication with a wireless communication module (e.g., a BLE beacon) installed in a hospital, a sensor module (not shown) that may sense the strength of a radio signal received from the wireless communication module installed in the hospital (e.g., a RSSI (Received Signal Strength Indication) sensor module), a location recognition module (not shown) that may acquire information on a location of the patient device 300 (e.g., a GPS module, an IMU (Inertial Measurement Unit), etc.), and the like.

For example, a wireless communication module (not shown) that may be included in the patient device 300 according to one embodiment of the invention may be assumed to be a wireless communication module with BLE technology, a WLAN (Wireless LAN) communication module, an ultrasonic communication module, an ACR (Access Control Router) module, or the like. However, it is noted that the configuration of the patient device 300 according the invention is not necessarily limited to the foregoing, and may be changed without limitation as long as the objects of the invention may be achieved.

Meanwhile, according to one embodiment of the invention, the patient device 300 may include an application to assist a patient to receive medical services according to the invention. The application may be downloaded from the main system 200 or an external application distribution server (not shown).

Next, according to one embodiment of the invention, the medical attendant device (or assistant medical attendant device) 400 is digital equipment that may function to connect to and then communicate with the main system 200, the patient device 300, or the hospital device (or hospital server) 500, and any type of digital equipment having a memory means and a microprocessor for computing capabilities, such as a smart phone and a tablet PC, may be adopted as the medical attendant device 400 according to the invention. Particularly, according to one embodiment of the invention, the medical attendant device 400 may be a wearable device such as smart glasses, a smart watch, a smart band, a smart ring, and a smart necklace.

Specifically, according to one embodiment of the invention, the medical attendant device 400 may include a communication module (not shown) for wireless communication with a wireless communication module (e.g., a BLE beacon) installed in a hospital, a sensor module (not shown) that may sense the strength of a radio signal received from the wireless communication module installed in the hospital (e.g., a RSSI (Received Signal Strength Indication) sensor module), a location recognition module (not shown) that may acquire information on a location of the medical attendant device 400 (e.g., a GPS module, an IMU (Inertial Measurement Unit), etc.), and the like.

For example, a wireless communication module (not shown) that may be included in the medical attendant device 400 according to one embodiment of the invention may be assumed to be a wireless communication module with BLE technology, a WLAN (Wireless LAN) communication module, an ultrasonic communication module, an ACR (Access Control Router) module, or the like. However, it is noted that the configuration of the medical attendant device 400 according the invention is not necessarily limited to the foregoing, and may be changed without limitation as long as the objects of the invention may be achieved.

Meanwhile, according to one embodiment of the invention, the medical attendant device 400 may include an application to assist a medical attendant to receive information for medical services according to the invention. The application may be downloaded from the main system 200 or an external application distribution server (not shown).

Next, according to one embodiment of the invention, the hospital device (or hospital server) 500 is digital equipment that may function to connect to and then communicate with the main system 200, the patient device 300, or the medical attendant device 400, and may control functions of the hospital device 500 or other hospital devices 500 connected to the hospital device 500, on the basis of information on dynamic matching between patients and medical attendants (or assistant medical attendants) performed by the main system 200.

Configuration of the Main System

Hereinafter, the internal configuration of the main system 200 according to the invention and the functions of the respective components thereof will be discussed.

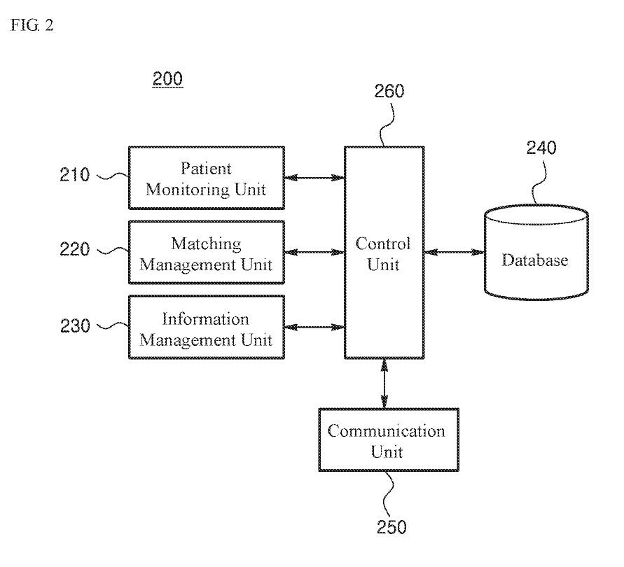

FIG. 2 specifically shows the internal configuration of the main system 200 according to one embodiment of the invention.

As shown in FIG. 2, the main system 200 according to one embodiment of the invention may comprise a patient monitoring unit 210, a matching management unit 220, an information management unit 230, a database 240, a communication unit 250, and a control unit 260. According to one embodiment of the invention, at least some of the patient monitoring unit 210, the matching management unit 220, the information management unit 230, the database 240, the communication unit 250, and the control unit 260 may be program modules to communicate with the patient device 300 or the hospital device (or hospital server) 500. The program modules may be included in the main system 200 in the form of operating systems, application program modules, and other program modules, while they may be physically stored in a variety of commonly known storage devices. Further, the program modules may also be stored in a remote storage device that may communicate with the main system 200. Meanwhile, such program modules may include, but not limited to, routines, subroutines, programs, objects, components, data structures, and the like for performing specific tasks or executing specific abstract data types as will be described below in accordance with the invention.

First, according to one embodiment of the invention, the patient monitoring unit 210 may function to determine an in-hospital status and a necessary medical service of a patient visiting a hospital, with reference to information on a location of the patient and information on a medical appointment or an electronic medical record (EMR) of the patient.

Here, according to one embodiment of the invention, the in-hospital status of the patient determined as above may be any one of “Waiting before treatment”, “Waiting during treatment”, “Under treatment”, and “Treatment completed”. Specifically, according to one embodiment of the invention, “Waiting before treatment” may indicate a status in which the patient is waiting at a desk or waiting room of the hospital; “Waiting during treatment” may indicate a status in which the patient is located at a chair or bed in a treatment room of the hospital but is waiting without being treated yet; “Under treatment” may indicate a status in which the patient is under treatment at a chair or bed in a treatment room of the hospital; and “Treatment completed” may indicate a status in which the patient has completed receiving treatment in the hospital. For example, when a patient A is located at a waiting room in the hospital and there is no new record in an electronic medical record of the patient A, it may be determined that the patient A is in the status of “Waiting before treatment.” As another example, when a patient B is seated on a first chair in a treatment room of the hospital, it may be determined that the patient B is in the status of “Waiting during treatment” or “Under treatment.” As yet another example, when a patient C is located at a waiting room in the hospital and there is a new record related to a scheduled treatment in an electronic medical record of the patient C, it may be determined that the patient C is in the status of “Treatment completed.”

Further, according to one embodiment of the invention, the necessary medical service of the patient may indicate a medical service that is estimated to be necessary for the patient, judging from the location of the patient and the medical appointment or electronic medical record of the patient. For example, when a patient A who is scheduled to receive gum treatment on Mar. 2, 2016 visits a hospital on Mar. 2, 2016, it may be determined that the necessary medical service of the patient A is gum treatment.

Meanwhile, according to one embodiment of the invention, information on the location of the patient and information on a location of a medical attendant or assistant medical attendant to be described below may be acquired from an in-hospital wireless communication module (e.g., a BLE beacon) that performs short-range wireless communication with the patient device 300 or the medical attendant device (or assistant medical attendant device) 400. Further, according to one embodiment of the invention, the information on the location of the patient or the information on the location of the medical attendant may be determined on the basis of information acquired from a location recognition module (e.g., a GPS module, an IMU, etc.) provided in the patient device 300 or the medical attendant device 400. However, the location recognition techniques applicable to the invention are not necessarily limited to the foregoing, and may be changed without limitation as long as the objects of the invention may be achieved.

Next, according to one embodiment of the invention, the matching management unit 220 may function to dynamically match the patient with at least one medical attendant or assistant medical attendant belonging to the hospital visited by the patient, with reference to information on the in-hospital status and necessary medical service of the patient determined as above, and information on an in-hospital status and a providable medical service of the medical attendant or assistant medical attendant. According to one embodiment of the invention, a matching relationship between the patient and the at least one medical attendant or assistant medical attendant may be periodically updated at a predetermined time interval, or may be aperiodically updated as the information referred to for the matching is changed.

Here, according to one embodiment of the invention, the in-hospital status of the medical attendant or assistant medical attendant may be determined on the basis of a location, a treatment schedule, a current treatment situation, and the like of the medical attendant or assistant medical attendant. For example, the in-hospital status of the medical attendant or assistant medical attendant may be any one of “Resting”, “Waiting for treatment”, and “Giving treatment”.

Specifically, according to one embodiment of the invention, the matching management unit 220 may determine treatment priority of the patient with reference to the information on the in-hospital status and necessary medical service of the patient. Further, according to one embodiment of the invention, the matching management unit 220 may match the patient with the at least one medical attendant or assistant medical attendant on the basis of the determined treatment priority of the patient, with reference to the information on the in-hospital status and providable medical service of the medical attendant or assistant medical attendant. Here, according to one embodiment of the invention, when there is an existing medical attendant or assistant medical attendant matched with the patient, the matching management unit 220 may replace the existing medical attendant or assistant medical attendant with a new medical attendant or assistant medical attendant.

More specifically, when it is determined that the in-hospital status of the patient is “Waiting before treatment” or “Waiting during treatment” and the waiting time or estimated waiting time of the patient exceeds a predetermined level, the matching management unit 220 may increase the treatment priority of the patient.

Here, according to one embodiment of the invention, one exemplary algorithm that may be used for matching a patient and a medical attendant or assistant medical attendant may be assumed to be a deep learning-based artificial intelligence algorithm that finds out an optimal match by performing learning on the basis of data on an in-hospital status of the patient, an in-hospital status of the medical attendant or assistant medical attendant, a matching relationship between the patient and the medical attendant or assistant medical attendant, a waiting time of the patient, and the like. Using such a deep learning-based artificial intelligence algorithm, a treatment pattern or treatment capability of a medical attendant to be matched according to the invention may be even recognized.

However, it is noted that the algorithm that may be used for matching a patient and a medical attendant or assistant medical attendant according the invention is not necessarily limited to the foregoing, and may be changed without limitation as long as the objects of the invention may be achieved.

Further, according to one embodiment of the invention, the matching management unit 220 may function to distribute information on a matching relationship between the patient and the at least one medical attendant or assistant medical attendant to the devices 400 of medical attendants or assistant medical attendants belonging to the hospital and at least one hospital device (or hospital server) 500 that manages medical resources of the hospital.

Next, according to one embodiment of the invention, the information management unit 230 may function to provide information for treatment of the patient to the device 400 of the at least one medical attendant or assistant medical attendant matched with the patient.

Specifically, according to one embodiment of the invention, the information provided to the device 400 of the medical attendant or assistant medical attendant may include notification information for calling the medical attendant or assistant medical attendant, or information required for proceeding with treatment of the patient matched with the medical attendant or assistant medical attendant (e.g., the patient's name, sex, age, waiting time, estimated treatment time, electronic medical record, consultation history, treatment approach, medicines or medical appliances required for treatment, etc.) More specifically, according to one embodiment of the invention, the know-how of a relatively skilled medical attendant or assistant medical attendant may be reflected in the information provided to the device 400 of the medical attendant or assistant medical attendant.

More specifically, according to one embodiment of the invention, the information provided to the device 400 of the medical attendant may include a variety of information such as information on a treatment pattern (or know-how) of the medical attendant or other medical attendant for each subdivided treatment item, information on deficient treatment items of the medical attendant as compared to other medical attendants, and information on new medical knowledge published at an academic conference or seminar.

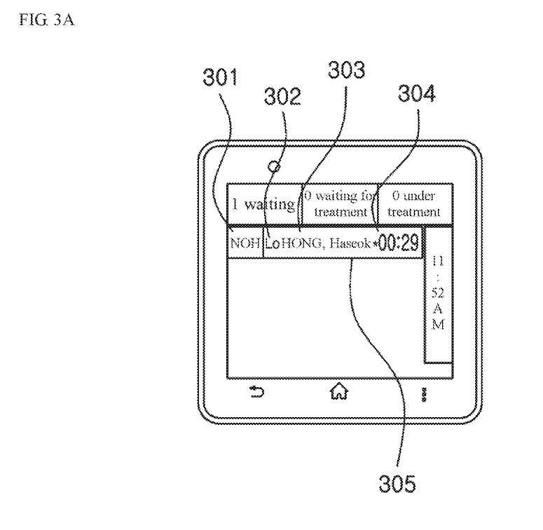

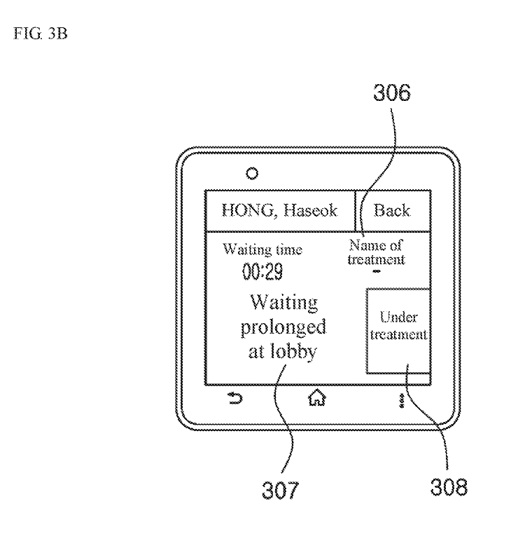

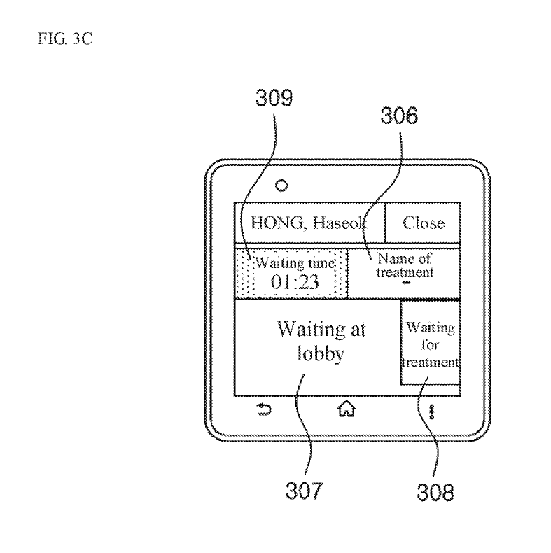

FIGS. 3A-3C illustratively show information provided to a device of a medical attendant or assistant medical attendant according to one embodiment of the invention.

First, referring to FIG. 3A, information on a name 303 of a patient currently visiting a hospital, a name 301 of a matched medical attendant or assistant medical attendant matched with the patient, a location 302 of the patient in the hospital, an in-hospital status 305 of the patient, a waiting time 304 of the patient, and the like may be provided to the device 400 of the medical attendant or assistant medical attendant. The above information may be periodically updated at a predetermined time interval, or may be aperiodically updated as the matching relationship is changed. Referring further to FIG. 3A, the information management unit 230 according to one embodiment of the invention may visualize at least a part of the provided information, thereby assisting the medical attendant or assistant medical attendant to intuitively understand the information. For example, a patient in the status of “Waiting before treatment” may be shown in white; a patient in the status of “Waiting during treatment” may be shown in green, a patient in the status of “Under treatment” may be shown in blue; and a patient whose waiting time is not less than ten minutes may be shown in red.

Next, referring to FIGS. 3B and 3C, detailed information on a specific patient (e.g., a necessary medical service (i.e., name of treatment 306)) may be provided to the device 400 of the medical attendant or assistant medical attendant, and the medical attendant or assistant medical attendant may personally change the in-hospital status of the patient by touching an icon 308 shown in relation to the patient on the device 400. Referring further to FIGS. 3B and 3C, when the waiting time of the patient exceeds a predetermined level (e.g., ten minutes, thirty minutes, one hour, or the like), information on the patient may be shown together with a background 309 shaded in red or other color, thereby providing a visual alarm to the medical attendant or assistant medical attendant.

Meanwhile, according to one embodiment of the invention, the patient monitoring unit 210 may function to assess a satisfaction level of the patient on the basis of information on an amount of time that the patient takes to stay in the hospital and receive treatment.

Meanwhile, according to one embodiment of the invention, the information management unit 230 may predict a change in sales of a medical institution from a treatment schedule specified by a state of matching between a patient and a medical attendant, and may provide information on the prediction to the hospital device 500, thereby providing useful information to management staff of the medical institution.

Meanwhile, according to one embodiment of the invention, the database 240 may store information on a location of a patient, medical attendant, or assistant medical attendant, information on a medical appointment of the patient, information on an electronic medical record of the patient, information on an in-hospital status of the patient, information on a necessary medical service of the patient, information on an in-hospital status of the medical attendant or assistant medical attendant, information on a providable medical service of the medical attendant or assistant medical attendant, information on a state of matching between the patient and the medical attendant or assistant medical attendant, treatment-related information to be provided to the device 400 of the medical attendant or assistant medical attendant, and the like. Although FIG. 2 shows that the database 240 is incorporated in the main system 200, the database 240 may be configured separately from the main system 200 as needed by those skilled in the art to implement the invention. Meanwhile, the database 240 according to the invention encompasses a computer-readable recording medium, and may refer not only to a database in a narrow sense but also to a database in a broad sense including file system-based data records and the like. The database 240 according to the invention may be even a collection of simple logs if one can search and retrieve data from the collection.

Next, the communication unit 250 according to one embodiment of the invention may function to enable data transmission/reception from/to the main system 200.

Lastly, the control unit 260 according to one embodiment of the invention may function to control data flow among the patient monitoring unit 210, the matching management unit 220, the information management unit 230, the database 240, and the communication unit 250. That is, the control unit 260 according to the invention may control data flow into/out of the main system 200 or data flow among the respective components of the main system 200, such that the patient monitoring unit 210, the matching management unit 220, the information management unit 230, the database 240, and the communication unit 250 may carry out their particular functions, respectively.

### Embodiments

Hereinafter, various embodiments for providing a medical service on the basis of matching between a patient and a medical attendant or assistant medical attendant according to the invention will be described in detail.

Conventionally, it is common that some of medical attendants and assistant medical attendants working in a hospital proceed with treatment of patients in order of visit or in an order determined by medical appointments, and the medical attendants and assistant medical attendants mainly carry out only the treatment of the patients under their charge, while they are roughly aware of information on waiting states or treatment states of the patients which may change every moment. Therefore, there is a limitation in reducing waiting times of the patients or improving the efficiency of treatment.

Further, when an unexpected situation occurs, e.g., when a patient visits the hospital later than his/her appointment time or when a treatment time for a patient becomes longer than expected, the waiting times of the patients to be subsequently treated become longer and complaints are received from the patients. The medical attendants or assistant medical attendants entirely bear the work burden of addressing and managing such a situation and thus cannot concentrate on the treatment, which may lead to a vicious circle of reduced treatment efficiency and increased patient complaints.

However, according to one embodiment of the invention, a patient may be dynamically matched with a medical attendant or assistant medical attendant belonging to a hospital, on the basis of information on an in-hospital status of the patient, a necessary medical service of the patient, an in-hospital status of the medical attendant or assistant medical attendant, and a providable medical service of the medical attendant or assistant medical attendant. Further, the device 400 of the medical attendant or assistant medical attendant may be provided with a variety of information for treatment of the patient matched with the medical attendant or assistant medical attendant, and information on waiting states of all patients located in the hospital, so that the medical attendant or assistant medical attendant may efficiently manage the waiting states and treatment states of the patients.

For example, it may be assumed that two medical attendants (i.e., doctors A and B) are working in a hospital and a total of nine patients are visiting the hospital, wherein patients a, b, c, d, e and f are located in a treatment room in the status of “Waiting during treatment” and patients l, m and n are located in a waiting room in the status of “Waiting before treatment”. Further, it may be assumed that in-hospital status, necessary medical services (i.e., treatments), and estimated treatment times of the above patients are as below.

Patient a (Waiting during treatment): deep anesthesia (5 min.)—crown removal (20 min.)

Patient b (Waiting during treatment): Caviton removal (1 min.)—root canal symptom check (1-2 min.)—root canal disinfection (5 min.)—Caviton restoration (1 min.)

Patient c (Waiting during treatment): ocular inspection for gum check—diagnosis (5 min.)

Patient d (Waiting during treatment): deep anesthesia (5 min.)—surgery preparation (10 min.)—surgery (45 min.)

Patient e (Waiting during treatment): treatment after surgery (1 min.)

Patient f (Waiting during treatment): simple anesthesia (2 min.)—gum treatment (8 min.)

Patient l (Waiting before treatment): Caviton removal (1 min.)—root canal symptom check (1-2 min.)—root canal disinfection (5 min.)—Caviton restoration (1 min.)

Patient m (Waiting before treatment): deep anesthesia (5 min.)—crown removal (20 min.)—impression taking (5 min.)—temporary tooth construction (20 min.)

Patient n (Waiting before treatment): treatment after surgery (1 min.)

In this case, according to one embodiment of the invention, the doctor A does not have to roughly determine the treatment order on the basis of inaccurate status information recognized by himself/herself or an assistant medical attendant, but may treat the patients matched with him/her according to notification information provided to the device 400 of the doctor A. Here, according to one embodiment of the invention, the notification information provided to the device 400 of the doctor A may include information on the matching with the patients and information required for treatment of the patients. Specifically, according to one embodiment of the invention, the doctor A may utilize the information provided to the device 400 of the doctor A so that he/she may treat other patients (i.e., the patients b, c, l and m) using spare time while treating the patient a.

First, according to one embodiment of the invention, the doctor A may give deep anesthesia for the patient a (5 min.) and carry out Caviton removal for the patient b (1 min.) and an ocular inspection for gum check—diagnosis for the patient c (5 min.) while the patient a rinses his/her mouth, according to the notification information provided to the device 400 of the doctor A.

Next, according to one embodiment of the invention, the doctor A may carry out crown removal for the patient a to an extent of 1/4 (5 min.) and carry out the remaining treatment for the patient b (i.e., root canal symptom check (1-2 min.)—root canal disinfection (5 min.)—Caviton restoration (1 min.)) while the patient a rinses his/her mouth, according to the notification information provided to the device 400 of the doctor A.

Next, according to one embodiment of the invention, the doctor A may carry out the crown removal for the patient a to an extent of 2/4 (5 min.), carry out Caviton removal for the patient l (1 min.) while the patient a rinses his/her mouth, carry out the crown removal for the patient a to an extent of 3/4 (5 min.) while the patient l rinses his/her mouth, and carry out the remaining treatment for the patient l (i.e., root canal symptom check (1-2 min.)—root canal disinfection (5 min.)—Caviton restoration (1 min.)) while the patient a rinses his/her mouth, according to the notification information provided to the device 400 of the doctor A.

Next, according to one embodiment of the invention, the doctor A may carry out the crown removal for the patient a to an extent of 4/4 (5 min.) to complete the crown removal, and give deep anesthesia for the patient m (5 min.) while the patient a rinses his/her mouth, according to the notification information provided to the device 400 of the doctor A.

Meanwhile, according to one embodiment of the invention, the doctor B who carries out treatment in parallel with the doctor A does not have to roughly determine the treatment order on the basis of inaccurate status information recognized by himself/herself or the assistant medical attendant, but may treat the patients matched with him/her (i.e., patients d, e, f and n) according to notification information provided to the device 400 of the doctor B.

Meanwhile, according to one embodiment of the invention, the assistant medical attendant may provide guidance on the treatment order, precautions before/after treatment, and the like to the patients who are in the status of “Waiting before treatment” or “Treatment completed” on the basis of notification information provided to the device 400 of the assistant medical attendant.

The embodiments according to the invention as described above may be implemented in the form of program instructions that can be executed by various computer components, and may be stored on a non-transitory computer-readable recording medium. The computer-readable recording medium may include program instructions, data files, data structures and the like, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler or the like, but also high-level language codes that can be executed by a computer using an interpreter or the like. The above hardware devices may be changed to one or more software modules to perform the processes of the present invention, and vice versa.

Although the present invention has been described in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method performed in a system for providing a medical service, the system comprising a patient monitoring unit and a matching management unit, and the method comprising the steps of:
the patient monitoring unit determining an in-hospital status and a necessary medical service of a patient visiting a hospital, with reference to information on a location of the patient and information on a medical appointment or an electronic medical record (EMR) of the patient; and
the matching management unit dynamically matching the patient with at least one medical attendant or assistant medical attendant belonging to the hospital, with reference to information on at least one of the in-hospital status of the patient, the necessary medical service of the patient, an in-hospital status of the medical attendant or assistant medical attendant, and a providable medical service of the medical attendant or assistant medical attendant,
wherein the in-hospital status of the patient is any one of “Waiting before treatment”, “Waiting during treatment”, “Under treatment”, and “Treatment completed”,
wherein the matching step comprises the steps of:
determining treatment priority of the patient with reference to information on the in-hospital status and necessary medical service of the patient; and
dynamically matching the patient with the at least one medical attendant or assistant medical attendant on the basis of the treatment priority of the patient, with reference to information on the in-hospital status and providable medical service of the medical attendant or assistant medical attendant, and
wherein in the matching step, the treatment priority of the patient is increased when the in-hospital status of the patient is “Waiting before treatment” or “Waiting during treatment” and a waiting time or estimated waiting time of the patient exceeds a predetermined level.

2. The method of claim 1, wherein the in-hospital status of the medical attendant or assistant medical attendant is determined on the basis of information on at least one of a location, a treatment schedule, and a current treatment situation of the medical attendant or assistant medical attendant.

3. The method of claim 1, wherein information on a location of the patient, medical attendant, or assistant medical attendant is determined on the basis of at least one of information acquired from at least one beacon that performs short-range wireless communication with a device carried by the patient, medical attendant, or assistant medical attendant, and information acquired from a location recognition module provided in the device carried by the patient, medical attendant, or assistant medical attendant.

4. The method of claim 1, wherein in the matching step, an existing medical attendant or assistant medical attendant matched with the patient is replaced with a new medical attendant or assistant medical attendant.

5. The method of claim 1, wherein the system further comprises an information management unit, and the method further comprises the step of:
the information management unit providing a device of the at least one matched medical attendant or assistant medical attendant with at least one of information on the matching, information on treatment of the patient, and notification information for calling the at least one matched medical attendant or assistant medical attendant.

6. A non-transitory computer-readable recording medium having stored thereon a computer program for executing the method of claim 1.

7. A system for providing a medical service, comprising:
a patient monitoring unit configured to determine an in-hospital status and a necessary medical service of a patient visiting a hospital, with reference to information on a location of the patient and information on a medical appointment or an electronic medical record (EMR) of the patient; and
a matching management unit configured to dynamically match the patient with at least one medical attendant or assistant medical attendant belonging to the hospital, with reference to information on at least one of the in-hospital status of the patient, the necessary medical service of the patient, an in-hospital status of the medical attendant or assistant medical attendant, and a providable medical service of the medical attendant or assistant medical attendant,
wherein the in-hospital status of the patient is any one of “Waiting before treatment”, “Waiting during treatment”, “Under treatment”, and “Treatment completed”,
wherein the matching management unit is configured to determine treatment priority of the patient with reference to information on the in-hospital status and necessary medical service of the patient, and to dynamically match the patient with the at least one medical attendant or assistant medical attendant on the basis of the treatment priority of the patient, with reference to information on the in-hospital status and providable medical service of the medical attendant or assistant medical attendant, and
wherein the matching management unit is configured to increase the treatment priority of the patient when the in-hospital status of the patient is “Waiting before treatment” or “Waiting during treatment” and a waiting time or estimated waiting time of the patient exceeds a predetermined level.

8. The system of claim 7, wherein the in-hospital status of the medical attendant or assistant medical attendant is determined on the basis of information on at least one of a location, a treatment schedule, and a current treatment situation of the medical attendant or assistant medical attendant.

9. The system of claim 7, wherein information on a location of the patient, medical attendant, or assistant medical attendant is determined on the basis of at least one of information acquired from at least one beacon that performs short-range wireless communication with a device carried by the patient, medical attendant, or assistant medical attendant, and information acquired from a location recognition module provided in the device carried by the patient, medical attendant, or assistant medical attendant.

10. The system of claim 7, wherein the matching management unit is configured to replace an existing medical attendant or assistant medical attendant matched with the patient with a new medical attendant or assistant medical attendant.

11. The system of claim 7, further comprising an information management unit configured to provide a device of the at least one matched medical attendant or assistant medical attendant with at least one of information on the matching, information on treatment of the patient, and notification information for calling the at least one matched medical attendant or assistant medical attendant.
